(19) United States
(12) Patent Application Publication
(10) Pub. No.: US 2016/0166213 A1
(43) Pub. Date: Jun. 16, 2016

(54) SELF-IDENTIFYING OXIMETRY SENSOR SYSTEM

(71) Applicant: NONIN MEDICAL, INC., North Plymouth, MN (US)

(72) Inventor: Philip O. Isaacson, Chanhassen, MN (US)

(21) Appl. No.: 14/436,825

(22) PCT Filed: Oct. 25, 2013
(86) PCT No.: PCT/US2013/066802

§ 371 Date: Apr. 17, 2015

Related U.S. Application Data
(60) Provisional application No. 61/718,831, filed on Oct. 26, 2012.

(51) Int. Cl. A61B 5/00; A61B 5/1495; A61B 5/145

(52) U.S. Cl. CPC A61B 5/742; A61B 5/14542; A61B 5/684; A61B 5/1495

(57) ABSTRACT

A system includes a plurality of sensors and a monitor. Each sensor of the plurality of sensors has a sensor output corresponding to a physiological measurement. Each sensor has a parameter associated with the sensor output. The monitor has a monitor processor, a display, and multiple channels. Each channel is configured to receive a sensor output from a sensor of the plurality of sensors. The monitor processor is configured to execute a set of instructions and configured to depict the physiological measurement using the display. The sensor output is configured using the parameter associated with the sensor output. The parameter corresponds to at least one of the channel associated with the sensor and a code received using the channel.

### PRIORITY APPLICATION

This application claims the benefit of priority to U.S. Provisional Application Ser. No. 61/718,831, filed Oct. 26, 2012, which is hereby incorporated by reference in its entirety.

### BACKGROUND

Regional oximetry, sometimes called tissue oximetry, provides a measure of oxygenation of blood in tissue. The measurement can provide important data as to the health of specific tissue. Equipment to monitor regional oximetry of a patient can provide information regarding tissue oxygenation for multiple tissue sites of a patient. For example, a patient considered to be at risk for compartment syndrome may be monitored using a number of external sensors with individual sensors located, for instance, at upper leg sites, at lower leg sites, and at additional sites on the patient arms. The individual sensors can be attached to the tissue site using a strap, an adhesive, a garment or by other means.

A system can include one or more sensors and a monitor. The monitor is in communicating with the sensors and can include a processor, a memory, a display and other elements to monitor the data or signal provided by the sensor. Currently available monitors are inadequate. For example, the costs and burden associated with managing a large number of sensors is sometimes excessive. In addition, the various sensors may be improperly affixed to the patient.

### OVERVIEW

The present subject matter relates to a self-identifying sensor system. One example includes a system having a sensor that, when plugged into a monitor, is configured to identify the part of a body on which the sensor is to be located. A display of the monitor is configured to indicate the location on the body of the patient for that sensor. For example, the display can include a depiction of a body and, upon detecting connection of a sensor, the display is configured to indicate the left side of a body and for another sensor, the display is configured to indicate the right side of a body. In one example, the various sensors are marked on an external surface to indicate that the sensor is designed for mounting at a particular part of the body.

An example of the present subject matter is tailored for monitoring compartment syndrome. Compartment syndrome is characterized by compression of nerves, blood vessels and muscles within a region, or a compartment, of a body. The compression leads to increased pressure in a muscle compartment and aggressive swelling. Aggressive swelling can restrict or cut off blood flow to a limb of a patient.

Compartment syndrome is commonly associated with the forearm and lower leg area and can be caused by bone fractures, ischemic reperfusion following injury, hemorrhage, vascular puncture, intravenous drug injection, casts, prolonged limb compression, crush injuries and burns. Compartment syndrome is often associated with battle injuries from explosive devices.

Treatment for compartment syndrome can include a variety of surgical procedures and in some cases, this can include limb amputation.

The present subject matter is directed at a system configured to detect and monitor tissue health. For example, one system can be used to monitor tissue oxygenation at multiple locations of a body and, as such, can be used for detecting and monitoring various conditions, including compartment syndrome.

Consider an example where a leg of a patient has suffered trauma, such as a crushing injury. The leg is in danger of swelling and loss of blood flow which may require treatment by amputation. The patient can be monitored using a system as described herein. The monitor can be coupled to individual sensors affixed on the injured leg as well as the uninjured leg and other portions of the patient. The sensors can provide regional oximetry measurements corresponding to the various regions of the body.

In one example, the system executes an algorithm that can read data encoded in the individual sensors and determines how to display the data from each particular sensor. If the sensor is encoded with information, then the system is able to ignore the channel coding (normally used to denote the type of sensor or region of the body), and instead, the coded information in the sensor is used to determine how the data is displayed on the monitor.

In one example, a first mode of operation corresponds to a situation in which the regional oximetry data from a sensor is labeled and displayed based on the channel to which the sensor is connected. In this instance, the sensors can be of the same type, appear to be identical, and yet the system is able to determine the proper display mode and labeling for each sensor. In various examples, the data is communicated wirelessly or by a wired connection.

In one example, a second mode of operation corresponds to a situation in which the sensor is encoded with sensor information corresponding to sensor calibration information, display information, and sensor location information. The information encoded in the sensor is determinative without regard for the channel or port to which the sensor is connected.

In various examples, the sensors are encoded with information that can be read by the monitor or the sensors are marked in a manner to visually indicate the location for placement on a patient.

An example of the present subject matter is configured to automatically switch over from one mode of operation to another mode of operation. One example is configured to measure and monitor regional oximetry, pulse oximetry, and patent ductis (arterialosis). Other sensors can also be used, including a sensor configured for temperature monitoring, blood pressure monitoring, or other physiological data.

In addition to compartment syndrome, other conditions may be monitored using an example of the present subject matter. As to regional oximetry, the patient may be monitored at a plurality of tissue sites over a period of time. The tissue sites may be monitored using various types of sensor assemblies affixed at widely distributed locations about the body of the patient. The sensor assemblies are each coupled to a monitor which provides data processing and displays information regarding the oximetry data.

The manual task associated with affixing multiple sensors to the patient and properly connecting the various sensors can be rather burdensome. In one example, multiple sensors are configured for use at a particular tissue site and require connection to a particular port (or channel) of the monitor in order to properly display the oximetry data from that sensor.

The problem associated with properly repositioning and reconnecting the monitor and the various sensor assemblies is addressed by an example of the present subject matter. For example, one instance includes a monitor having a plurality of connector pods. Each connector pod can be coupled to a sensor assembly. The sensor assembly provides oximetry data as well as the location, type, and calibration information is provided automatically.

The problem of managing a regional oximetry monitor and the various associated sensors can be addressed by an example of the present subject matter. One example includes an oximetry monitor having a plurality of connector pods. The pods are coupled to the monitor and each pod is configured to couple with any of a number of sensor assemblies.

In one example, each connector pod is sensor agnostic. For example, a first sensor can include optical elements configured for monitoring tissue oximetry in a lower leg region and a second sensor can include optical elements configured for monitoring tissue oximetry in an upper arm region. A connector pod of the present subject matter can be connected with either the first sensor or the second sensor. Without regard for which sensor is connected, the monitor is configured to read data encoded in the sensor, determine a proper location for display and scaling of the data and then depict the data accordingly.

This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1A illustrates a diagram of a system according to one example.

FIG. 1B illustrates a block diagram of a monitor, according to one example.

FIG. 2 illustrates a diagram including a sensor and a pod, according to one example.

FIG. 3 illustrates a block diagram of a sensor, according to one example.

FIG. 4 illustrates a block diagram of a pod, according to one example.

FIG. 5 illustrates a method, according to one example.

### DETAILED DESCRIPTION

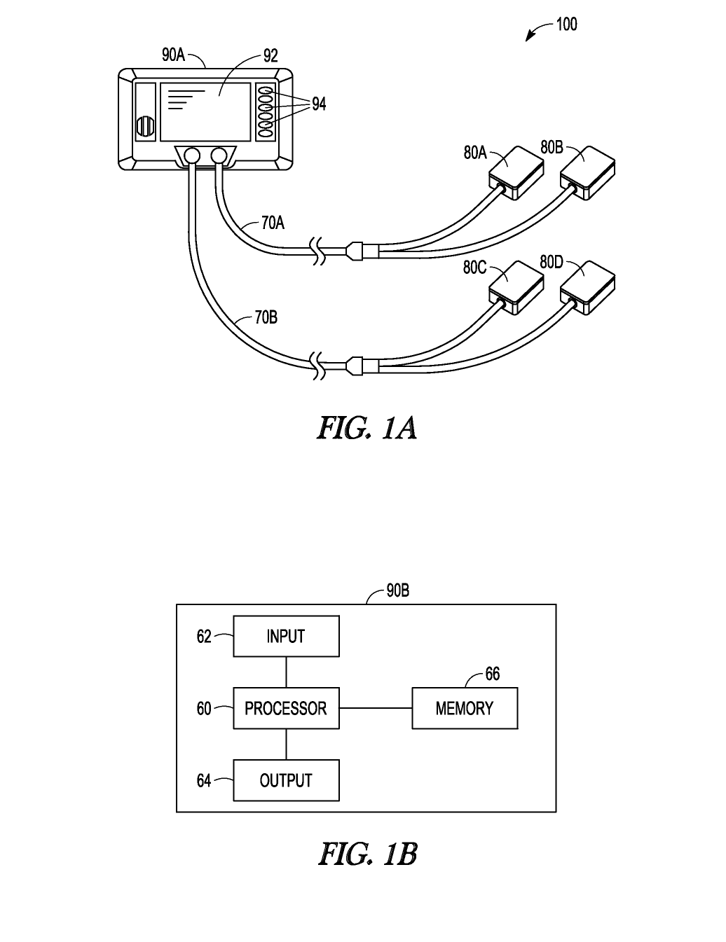

FIG. 1A illustrates a diagram of system 100 according to one example. The example illustrated includes monitor 90A and a plurality of pods (here shown as pod 80A, pod 80B, pod 80C, and pod 80D) coupled by a plurality of trunk cables (trunk cable 70A and trunk cable 70B). Monitor 90A includes visual display 92 and a plurality of user operable controls 94 located on a front panel. In the example shown, monitor 90A is coupled to two trunk cables 70A and 70B. Trunk cables 70A and 70B carry electrical signals between monitor 90A and pods 80A-80D. In various examples, each trunk cable (such as trunk cable 70A), or each pod (such as pod 80A), is associated with a particular channel of the monitor.

The figure illustrates monitor 90A coupled to the pod (such as pod 80A) by a wired communication link (such as trunk cable 70A); however, other examples include a wireless communication link between monitor 90A and the pod (such as pod 80A).

FIG. 1B illustrates a block diagram of monitor 90B. In the example shown, monitor 90B includes processor 60 coupled to input module 62, output module 64, and memory 66. Processor 60 can include an analog or digital processor configured to execute instructions or implement an algorithm. Memory 66 can provide storage for instructions as well as storage for measured data, for parameter values, for calibration information, and for other data. Memory 66 can include a look up table. Input module 62 can include a user-operable touch-screen, a keypad, a switch, a mouse controller, a network interface, or other type of input device. Output module 64 can include a printer, a display, a speaker, a network interface, or other type of output device. Monitor 90B can be implemented using a digital or analog circuit.

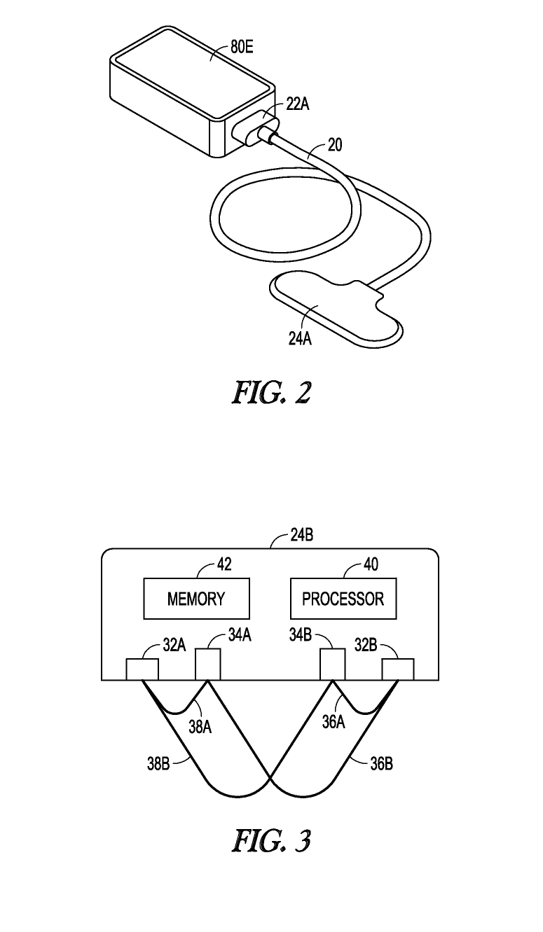

FIG. 2 illustrates sensor 24A and pod 80E, according to one example. In the figure, pod 80E is coupled by sensor cord 20 to sensor 24A. Sensor cord 20 is connected to pod 80E by sensor connector 22A. Sensor cord 20 enables unidirectional communication or bidirectional communication between sensor 24A and pod 80E. In one example, sensor cord 20 carries the sensor output signal to pod 80E and carries command signals (instructions) to sensor 24A.

Pod 80E is connected to a monitor (such as monitor 90A or 90B; not shown in this figure). Sensor 24A, in the example shown, is configured for bonding to a tissue surface. The figure illustrates sensor 24A coupled to pod 80E by a wired communication link (here shown as sensor cord 20); however, other examples include a wireless communication link between sensor 24A and pod 80E.

FIG. 3 illustrates a block diagram of sensor 24B, according to one example. In this example, sensor 24B includes processor 40, memory 42, first emitter 32A and second emitter 32B, and first detector 34A and second detector 34B. Emitters 32A and 32B and detectors 34A and 34B are distributed about a tissue contact surface of sensor 24B. Emitters 32A and 32B are configured to emit optical energy at wavelengths configured for measuring regional oximetry or pulse oximetry. Detectors 34A and 34B are configured to generate an output signal corresponding to detected optical energy corresponding to transmitted or reflected light energy. The optical elements (including emitters 32A and 32B and including detectors 34A and 34B) are spaced apart at distances corresponding to various depths of optical light penetration into the tissue. In the example shown, the multiple light paths (paths 36A, 36B, 38A and 38B) provide data that is useful for calibrating the sensor output.

In one example, sensor memory 42 provides storage for encoded data. In one example, encoded information is stored in the form of a component value, such as a resistor value, a capacitor value, or other component value.

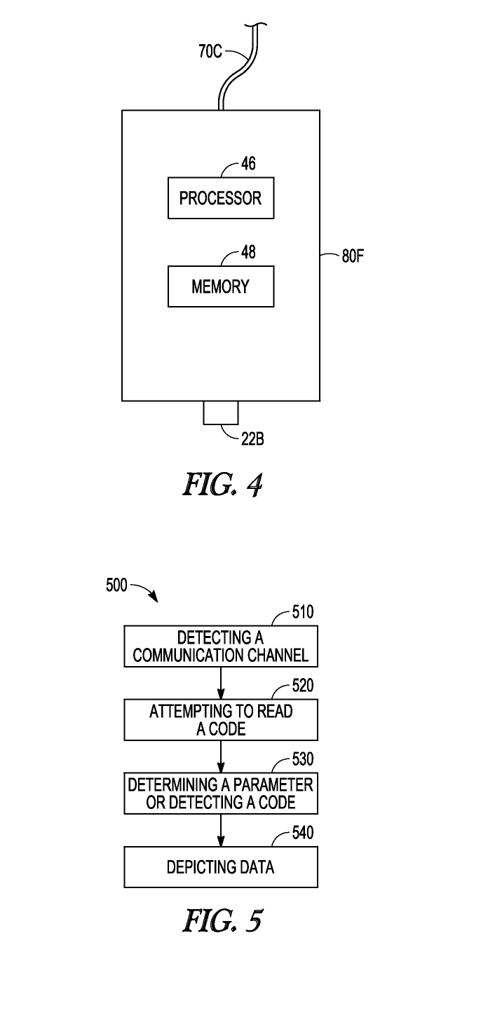

FIG. 4 illustrates a block diagram of pod 80F, according to one example. In the figure, pod 80F includes processor 46 and memory 48. Pod 80F includes sensor connector 22B and, in the example shown, includes trunk cable 70C to connect with a monitor (not shown). Pod processor 46 is configured to receive a sensor output signal, access memory contents, and provide calibrated sensor data to the monitor via trunk cable 70C. Memory 48 can include a look up table or values determined by a sensor connected to sensor connector 22B. In one example, pod processor 46 is configured to read encoded information of the sensor.

FIG. 5 illustrates method 500 according to one example of the present subject matter. At 510, method 500 includes detecting a communication channel, between, for example, a sensor and a monitor. The monitor can have a plurality of channels and each channel is configured for communicating between one sensor (of a plurality of sensors) and the monitor. At 520, method 500 includes attempting to read a code associated with the sensor. In one example, the code is received from the sensor. At 530, method 500 includes, in the absence of a code (or in the event of failure to read a code), determining a parameter for the sensor based on the communication channel. The parameter can be derived from the sensor type or sensor. The parameter, for example, can indicate that the sensor is tailored for use on the lower leg region of a body. In addition, method 500 includes, upon detecting the code, determining the parameter for the sensor based on the stored code. The parameter may correspond with a portion of the monitor display in which the sensor output is to be visually depicted. The parameter can correspond to a target site on a body and the target site can be depicted on a representation of the body. For example, the display can depict a silhouette of a body and indicate that the sensor is intended for coupling with the body at a particular location on the silhouette. In addition, the parameter can indicate that a particular calibration coefficient is to be applied to the data received from the sensor.

At 540, method 500 includes depicting data from the sensor on a display of the monitor. The data is displayed as a function of the parameter. For example, the data can be depicted on the monitor display according to the parameter.

Other configurations are also contemplated. For example, detecting the communication channel can include detecting a wired connection or detecting a wireless connection. In one example including a wireless connection, the sensor is battery-operated. In one example, attempting to read the code includes determining a value associated with a passive element of the sensor. For example, a passive element of the sensor (such as a resistor value) is determined by a voltage drop or change in current. Attempting to read the code can include determining a value associated with an active element of the sensor. The active element can include one or more digital elements, such as a value stored in a memory including a transistor or other semiconductor device. In one example, determining the parameter includes determining calibration information, determining a location on the display, determining a label for the display, or determining a body location associated with the sensor. More than one of the foregoing can be determined. For example, the method can include determining a body location associated with the sensor and include depicting the body location using the display. In one example, the method includes determining a classification for the sensor and depicting the classification.

### VARIOUS NOTES & EXAMPLES

Various examples of the present subject matter are configured to reduce the burden associated with using a monitor and a plurality of sensors. For example, a patient at risk for compartment syndrome may have a large number of sensors coupled to various sites on the body. The sensors can provide data concerning tissue (regional) oximetry, pulse oximetry, temperature, blood pressure, and other physiological measurements. Under certain circumstances, such as that associated with certain imaging modalities, the sensors and the monitor are temporarily disconnected. Following such procedures, the caregiver is burdened with the task of properly reconnecting each sensor to the correct pod and configuring the monitor to properly display the measured results.

Using an example of the present subject matter, the caregiver has multiple ways of establishing the proper connections to the monitor. First, the caregiver can sequentially attach each sensor cord to a pod and, by observing the display on the monitor; the caregiver can discern the tissue site for placement of that particular sensor. In addition, a caregiver can randomly connect a sensor with a pod and allow the system to discover and determine the parameters associated with that sensor. For example, a code embedded in the sensor can be read and interpreted by the monitor to determine the type of sensor, the tissue site associated with the sensor, the region of the display for which that data is to be displayed, the calibration information for that display, and the legend and scaling suitable for that sensor. Without regard for the type of sensor connected, one example of the present system can automatically switch from one mode of operation to another mode of operation.

In one example, a sensor has external markings to indicate the tissue site for coupling with the body of the patient. For example, a sensor body can be marked to denote the left, lower leg or marked to denote the right forearm.

One example of the present subject matter is configured to reduce the criticality as to selection of the sensor site and selection of the connector to which a particular sensor assembly is coupled. In one example, at least one of the sensor assembly or the connector pod is encoded to ensure that the sensor data is properly displayed and the sensor itself can be attached with increased tolerance for variations.

The monitor, which can be located near the patient or in a remote location, can be configured to read a code to determine a type of sensor connected at a port. The sensor can be identified without regard for the system channel (or pod) to which the sensor is connected.

The monitor can be configured to read a code from the sensor to determine a location on a display without regard for the system channel to which the sensor is connected.

One example of the system includes a monitor having processor, a display, and a memory. The monitor can include, or be connected to a printer. In addition, the monitor can be coupled to a network to enable communication and data exchange with remote devices that are also coupled to the network.

In one example, the monitor is connected to multiple sensors, with each sensor separately connected to a port of the monitor. A port can include an electrical connector affixed directly to the monitor or can include a connector (or pod) coupled to the monitor by an electrical cord.

Each port is associated, in one-to-one relation, with a sensor, and in relation to compartment syndrome, each sensor provides regional oximetry data for a separate compartment.

In one example, rather than requiring the caregiver to accurately plug a particular sensor into a particular pod, the caregiver can, instead, merely plug each sensor into a randomly selected pod. The system is configured to determine the differences between the various sensors and determine location and manner of display for each sensor connected to a monitor.

In one example, if each sensor is coded, then channel coding information (that is, information associated with a particular pod of the monitor) is ignored and the sensor code will be used for determining display characteristics.

A problem associated with properly repositioning and reconnecting the monitor and the various sensor assemblies is addressed by an example of the present subject matter. For example, one instance includes a monitor having a plurality of connector pods and each connector pod can be coupled to a sensor. The sensor provides oximetry data as well as information concerning the tissue site location, the sensor type, and sensor calibration information.

As noted earlier, monitoring and treatment of compartment syndrome many entail monitoring many sites. When sensors are initially placed, or later disturbed, it is important to position each sensor at the proper location and from time to time, reconnect each sensor to the proper port of the monitor so that meaningful data can be collected.

Among the challenges facing the caregiver are the problems of determining where to position the sensor on the body. In one example, the sensor body is marked with body location information. The sensor can be connected to any pod and the caregiver can read the monitor display which indicates the intended body location. In one example, the caregiver can select a port for connecting a sensor to the monitor. The sensor is marked for use at a particular location on the body. The sensor can be connected with any pod and the monitor reads the data encoded in the sensor and automatically configures the monitor to properly display the sensor information.

Two modes of operation can be described. In a first mode, the sensors are distributed on the body at known location (either the sensor is externally marked as to the location or the sensor is already positioned at the appropriate tissue site. Any pod can be selected for any sensor and the calibration info, display location information is determined based on the sensor type. The sensor is encoded with self-identifying information to allow the monitor to properly generate and display the physiological measurement. The monitor can read the self-identifying encoded information of the sensor and automatically adjust calibration in order to properly display the results.

In a second mode of operation, the sensor site is not yet determined or established (sensor is not yet placed on body or the sensor is not externally marked for a particular location). In this example, the challenge is to find where to place the sensor on the body and how to display the data properly. In this example, the caregiver can plug the sensor into any port and the monitor will automatically display the proper site location and provide proper calibration (sensor is encoded with information).

An example of the present subject matter can automatically switch over from one mode of operation to another mode of operation.

The sensor can be encoded with information in various ways, including in a memory or in a component value. In addition, the information can be encoded and stored in a location within a connecting cable, within a connector, or within the sensor housing itself. The tissue site information can be encoded in a digital or analog form.

A monitor, when connected with the sensor, is configured to decode the tissue site information associated with the sensor. Data displayed on the monitor can include the tissue site information associated with a particular pod to which the sensor is connected. For example, after connecting a sensor to a pod of the monitor, the display of the monitor indicates the site location for that particular sensor. This can aid the technician in placing the sensor at the proper site on the patient. For instance, the sensor can be encoded to indicate that it is tailored for use at a lower calf location on a patient and when connected to a monitor (via a pod), the display of the monitor indicates the lower calf location in a manner to facilitate placement of the sensor at that indicated location.

As such, the monitor is configured to display or indicate a tissue site corresponding to the tissue site information encoded in the sensor.

In one example, the sensors is labeled, marked, or configured in a manner that corresponds to the tissue site location. For example, a code can be printed, raised, or embossed on the sensor housing to indicate a sensor location on the patient. In addition, the sensor can be electrically encoded with the tissue site information.

The present subject matter can be configured to monitor for a medical condition known as patent ductis arteriosus. A suitably configured array of sensors can be fitted to the patient and coupled to a monitor to allow continuous monitoring of tissue oximetry at various sites of the patient's body.

Each of these non-limiting examples can stand on its own, or can be combined in any permutation or combination with any one or more of the other examples.

### EXAMPLES

### 1. A System Comprising:

a plurality of sensors, each sensor having a sensor output corresponding to a physiological measurement and a parameter associated with the sensor output; and

a monitor having a monitor processor, a display, and having multiple channels, wherein each channel is configured to receive a sensor output from a sensor of the plurality of sensors, the monitor processor configured to execute a set of instructions and configured to depict the physiological measurement using the display, the sensor output configured using the parameter associated with the sensor output, the parameter corresponding to at least one of the channel associated with the sensor and a code received using the channel.

2. The system of example 1 wherein at least one sensor includes a regional oximetry sensor.
3. The system of example 1 wherein a channel includes a connector pod coupled to the monitor, the connector pod having a pod processor configured to generate data corresponding to the sensor output.
4. The system of example 1 wherein the pod processor is configured to apply a calibration adjustment to the sensor output.
5. The system of example 1 wherein the monitor processor is configured to read encoded data corresponding to the parameter.
6. The system of example 1 wherein the monitor processor is configured to generate a time wise view of the physiological measurement using at least one of the code and the parameter.
7. A method comprising:

detecting a communication channel between a sensor and a monitor, the monitor having a plurality of channels, each channel configured for communicating between one sensor of a plurality of sensors and the monitor;

attempting to read a code associated with the sensor, the code received from the sensor;

in the absence of a code, determining a parameter for the sensor based on the communication channel; and

upon detecting the code, determining the parameter for the sensor based on the code; and

depicting data from the sensor on a display of the monitor wherein the data is displayed using the parameter.

8. The method of example 7 wherein detecting the communication channel includes detecting a wired connection.
9. The method of example 7 wherein attempting to read the code includes determining a value associated with a passive element of the sensor.
10. The method of example 7 wherein attempting to read the code includes determining a value associated with an active element of the sensor.
11. The method of example 7 wherein determining the parameter includes one of determining calibration information, determining a location on the display, determining a label for the display, and determining a body location associated with the sensor.
12. The method of example 7 further including determining a body location associated with the sensor and depicting the body location.
13. The method of example 7 further including determining a classification for the sensor and depicting the classification.
14. A method comprising:

accessing a code stored in a sensor, the sensor in communication with a monitor, the monitor having a display, the code having a value corresponding to a parameter associated with the sensor;

executing instructions using a processor of the monitor to determine a measure of oximetry based on a signal received from the sensor assembly and based on the value; and

displaying the measure of oximetry on the display using the code.
15. The method of example 14 wherein the value corresponds to display information for the sensor assembly.
16. The method of example 14 wherein the code corresponds to a location on the display of the monitor.
17. The method of example 14 further including indicating a sensor type on the display, the sensor type determined based on the code.
18. A system comprising:

a monitor having a display, the monitor configured to graphically depict a plurality of physiological measurements in a plurality of portions of the display; and

a plurality of pods coupled to the monitor, wherein each pod is configured to provide data corresponding to a sensor associated with that pod and configured to provide a code to the monitor, the code having a value and the code corresponding to a selected portion of the display, the data including a signal corresponding to the physiological measurement, the value determined by at least one of the pod and the associated sensor.

19. The system of example 18, wherein the pod includes a memory, the value determined by the memory.
20. The system of example 18, wherein the sensor includes a memory, the value determined by the memory.
21. The system of example 18, wherein the sensor includes a visible marking.
22. The system of example 18, wherein the visible marking corresponds to at least one of a tissue site and a sensor type.
23. The system of example 18, wherein the physiological measurement corresponds to regional oximetry.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations.

The claimed invention is:

1. A system comprising:
a plurality of sensors, each sensor having a sensor output corresponding to a physiological measurement and a parameter associated with the sensor output; and
a monitor having a monitor processor, a display, and having multiple channels, wherein each channel is configured to receive a sensor output from a sensor of the plurality of sensors, the monitor processor configured to execute a set of instructions and configured to depict the physiological measurement using the display, the sensor output configured using the parameter associated with the sensor output, the parameter corresponding to at least one of the channel associated with the sensor and a code received using the channel.

2. The system of claim 1 wherein at least one sensor includes a regional oximetry sensor.

3. The system of claim 1 wherein a channel includes a connector pod coupled to the monitor, the connector pod having a pod processor configured to generate data corresponding to the sensor output.

4. The system of claim 1 wherein the pod processor is configured to apply a calibration adjustment to the sensor output.

5. The system of claim 1 wherein the monitor processor is configured to read encoded data corresponding to the parameter.

6. The system of claim 1 wherein the monitor processor is configured to generate a time wise view of the physiological measurement using at least one of the code and the parameter.

7. A method comprising:
detecting a communication channel between a sensor and a monitor, the monitor having a plurality of channels, each channel configured for communicating between one sensor of a plurality of sensors and the monitor;
attempting to read a code associated with the sensor, the code received from the sensor;
in the absence of a code, determining a parameter for the sensor based on the communication channel; and
upon detecting the code, determining the parameter for the sensor based on the code; and
depicting data from the sensor on a display of the monitor wherein the data is displayed using the parameter.

8. The method of claim 7 wherein detecting the communication channel includes detecting a wired connection.

9. The method of claim 7 wherein attempting to read the code includes determining a value associated with a passive element of the sensor.

10. The method of claim 7 wherein attempting to read the code includes determining a value associated with an active element of the sensor.

11. The method of claim 7 wherein determining the parameter includes one of determining calibration information, determining a location on the display, determining a label for the display, and determining a body location associated with the sensor.

12. The method of claim 7 further including determining a body location associated with the sensor and depicting the body location.

13. The method of claim 7 further including determining a classification for the sensor and depicting the classification.
